(19) United States
(12) Patent Application Publication
(10) Pub. No.: US 2003/0124191 A1
(43) Pub. Date: Jul. 3, 2003

(54) Use of an immediate-release powder in pharmaceutical and nutraceutical compositions

(76) Inventors: Jerome Besse, Listrac Medoc (FR); Laurence Besse, Listrac Medoc (FR)

Correspondence Address: Michael L. Kenaga, Piper Marbury Rudnick & Wolfe, P.O. Box 64807, Chicago, IL, 60664-0807, US

(21) Appl. No.: 10/106,923

(22) Filed: Mar. 25, 2002

(30) Foreign Application Priority Data
Dec. 27, 2001 (FR) 0116934

(51) Int. Cl. A61K 31/7048; A61K 31/56; A61K 31/522; A61K 31/4965; A61K 31/445; A61K 31/573; A61K 31/496; A61K 31/4178; A61K 31/135

(52) U.S. Cl. 424/489; 514/182; 514/177; 514/178; 514/29; 514/649; 514/317; 514/343; 514/263.35; 514/554; 514/629; 514/263.33; 514/397; 514/255.04

(57) ABSTRACT

The present invention relates to the use of a powder comprising at least one active substance, at least one surfactant, at least one wetting agent and at least one diluent, for preparing a pharmaceutical or nutraceutical composition, this composition allowing rapid and immediate release of the active substance.

The present invention relates to the use of an immediate-release powder for buccal application, intended for the preparation of pharmaceutical or nutraceutical compositions.

The use according to the invention of a powder for preparing a pharmaceutical or nutraceutical composition allows a rapid release (or “flash”) of the active substance when the composition comprising it is administered mucosally.

Pharmaceutical forms which allow rapid release of an active substance are already known. They are tablets of the “lyoc” type or tablets which disintegrate rapidly in the mouth, such as for example the FLASHTAB® (ETHYPHARM) or SOBLET® technology, or film-type systems provided in the form of a “wafer”, i.e. films for buccal application which allow more or less rapid dissolution of the active substances.

This being so, these two pharmaceutical forms have several drawbacks. The tablets suffer from a significant friability, which makes them delicate to handle, and, moreover, their disintegration time is very often longer than 10 seconds. The films are difficult to apply due to their very small thickness. In addition, the two pharmaceutical forms suffer from a major drawback in that they allow only a relatively low load of active substance, diverse and varied excipients being required for their structural integrity.

The Applicant Companies have therefore sought to develop a pharmaceutical form which can overcome the drawbacks encountered by the prior formulations.

They have thus succeeded in developing a powder, the use of which in a pharmaceutical or nutraceutical composition allows rapid and immediate release of the active substance alone or in combination, when said composition is administered buccally.

For the purpose of the present invention, the expression “rapid and immediate release” is intended to mean release of all of the active substance(s) in less than 30 seconds, preferably less than 15 seconds and even more preferentially in less than 10 seconds.

The powder used according to the invention, unlike the tablets and films of the prior art, is delicate neither in terms of its handling nor in its application. It also allows a considerable active substance load. Specifically, the load of active substances per dose unit can be considerably greater than the 20 mg imposed in particular by the technology of the films of the “wafer” type or equivalent.

The use of the powder according to the present invention therefore has many advantages compared to the known pharmaceutical forms in the prior art.

Thus, the present invention relates to the use of a powder comprising at least one active substance, at least one surfactant, at least one wetting agent and at least one diluent, for preparing a pharmaceutical or nutraceutical composition, this composition allowing rapid and immediate release of the active substance when it is administered mucosally.

The active substances of the powder used according to the invention may be selected from those conventionally used in the following pharmacotherapeutic families: allergology, anaesthesia/reanimation, cancerology and haematology, cardiology and angiology, contraception and interruption of pregnancy, dermatology, endocrinology, gastroenterohepatology, gynaecology, immunology, infectiology, metabolism and nutrition, neurology/psychiatry, ophthalmology, otorhinolaryngology, pneumology, rheumatology, stomatology, toxicology, and urology/nephrology, and also from analgesics and antispasmodics, anti-inflammatory agents, contrast products used in radiology, haemostatics, and blood treatment products and derivatives.

Advantageously, the active substances may be selected from the group consisting of the active substances which cross the mucosal barrier and reach the systemic circulation, such as cyproterone acetate, Δ-4-androstenedione, 3-keto-desogestrel, desogestrel, gestodene, oestradiol and derivatives thereof, norethisterone acetate, progesterone, testosterone, dihydrotestosterone, trinitrine, fentanyl, nitroglycerine, nicotine (nicotine S(−)), scopolamine, clonidine, isosorbide dinitrate, laevonorgestrel in combination with ethinyl oestradiol or with oestradiol, androstanolone, alclometasone dipropionate, phloroglucinol, molsidomine, and combinations thereof.

They may also be selected from the active substances which cross the mucosal barrier and have a localized action, such as: acetazolamide, acyclovir, adapalene, alclomethasone dipropionate, amcinonide, ameline, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromicin, flumetasone pivalate, fluocinolon acetonide, fluocinodine, fluocortolone, fluocortolone hexanoate, fluocortolone pivalate, hydrocortisone, hydrocortison acetate, ibacitabine, ibuprofen, imiquimod, ketoconazole, ketoprofen, lidocaine, metronidazole, miconazole nitrate, minoxidil, nifluminic acid, penciclovir, benzoyl peroxide, piroxam, iodinated povidone, promestriene, pyrazinobutazone, roxithromycin, sulphacetamide, triamcinolone, tazarotene, tretinoin and isotretinoin, triclocarban, vidarabine monophosphate and combinations thereof.

They may also be selected from the following active substances: β-3-adrenergic agonist, growth hormone, oxybutinin, buprenorphine, pergolide, nestorone, 7α-methyl-19-nortesterone, mecamylamine, salbutamol, selegiline, buspirone, ketotifen, lidocaine, ketorolac, eptazocine, insulin, α-interferon, prostaglandins, 5-aminolevulinic acid, benzodiazepine alprozolam, diclofenac, fenoprofen, flubiprofen, ketoprofen, methyl phenidate, miconazole, piroxicam, bruprenorphine, dexmedetomidine, prazosin (α-adrenergic antagonist), alprostadil, tulobuterol (β-adrenergic agonist), ethinyl oestradiol+norelgestromin, physostigmine, medindolol (α-adrenergic agonist), rotigotine (dopamine D2 antagonist), thiatolserine and combinations thereof.

They may also be selected from the following active substances: Esomeprazole, Melagatran (in the case of thrombosis), Rosuvastatin, Ezetimide, Pitavastatin (hyperlipidaemia), Mitiglinide (type II diabetes), Cilomilast, Viozan (asthma), Aripipazole (psychiatry), Omapatrilat (hypertensive), Orzel (cancerology), Caspofongin acetate, Voriconazole (infections), new COX inhibitors such as Etoricoxib (inflammation), Valdecoxib (arthritis) and Parecoxib, Substance P antagonist (depression), Darifenacin (urology), Eletriptan (migraine), Alosetron, Tegaserod, Capravirine (HIV) and combinations thereof.

The powder used according to the invention may contain one or more active principles in combination with one another.

For nutraceutical applications, the active substance may be chosen from the list of raw materials authorized as food supplements, such as, for example, from the group consisting of vitamins, mineral salts, brewer's yeast, etc.

According to a preferential embodiment of the powder according to the invention, the active substances are micronized before being mixed with other ingredients. It is also possible to mix the non-micronized active substance with the other ingredients of the powder and then to micronize the final mixture. This promotes rapid (by increasing the surface area of contact with the buccal cavity) and homogeneous release of the active substance. Moreover, systems for spraying powder are particularly suitable for spraying micronized products.

The powder used according to the invention may also comprise one or more surfactants, preferably non-ionic surfactants, such as polyoxyethylene sorbitan (fatty acid ester), polyoxyethylene alkyl ether, the polyoxyethylene derived from castor oil, and mixtures thereof.

When needed, this powder may also comprise a wetting agent selected from the group consisting of polyols such as sorbitol, glycerol or PEG, and mixtures thereof.

The powder used according to the invention may also comprise a binding agent selected from the group consisting of acacia, alginic acid, sodium carboxymethylcellulose, microcrystalline cellulose, dextrins, ethylcellulose, gelatin, glucose, guar gum, hydroxypropylmethylcellulose, methylcellulose, polyethylene oxide, povidone, pregelatinized starch, and mixtures thereof.

The powder used according to the invention may also comprise a diluent selected from the group consisting of sodium or calcium carbonate or bicarbonate, sucrose, mannitol, xylitol, sorbitol, lactose, microcrystalline cellulose or cellulose powder, starch and derivatives thereof, dibasic calcium phosphate, tribasic calcium phosphate, calcium sulphate, dextrates, dextrins, dextrose excipients, fructose, kaolin, lactitol and mixtures thereof.

The powder used according to the invention may also comprise a penetration enhancer which may be selected from the group consisting of aliphatic fatty acid esters such as isopropyl myristate, fatty acids such as oleic acid; alcohols or polyols, such as ethanol, propylene glycol or polyethylene glycol; the components of essential oils and terpene derivatives (such as eugenol, geraniol, nerol, eucalyptol or menthol); surfactants; moisturizers such as glycerol or urea; keratolytic agents, such as alpha-hydroxy acids, 23-lauryl ether, aprotinin, azone, benzalkonium chloride, cetylpyridinium chloride, cetyltrimethylammonium bromide, cyclodextrins, dextran sulphate, lauric acid, lysophosphatidylcholine, menthol, methoxysalicylate, methyl oleate, oleic acid, phosphatidylcholine, polyoxyethylene, polysorbate 80, sodium EDTA, sodium glycocholate, sodium glycodeoxycholate, sodium lauryl sulphate, sodium salicylate, sodium taurocholate, sodium taurodeoxycholate, sulphoxides and alkyl glycosides.

According to a preferential embodiment of the powder used according to the invention, it has a particle size of between 0.01 μm and 1000 μm, preferably between 0.1 μm and 100 μm, and even more preferentially between 1 μm and 50 μm.

The composition containing the powder used according to the invention is administered mucosally. It may be applied, for example, on the buccal mucous membrane, the nasal mucous membrane or the vaginal mucous membrane, and also sublingually.

Advantageously, the composition comprising the powder used according to the invention is in a dry form packaged in a spray or in the form of a sachet. These formulations allow a precise dose of active material to be delivered easily.

All the methods known to those skilled in the art may be used in the context of producing the powder used according to the invention.

As an example of a method for preparing a powder, mention may be made of: wet or dry granulation, preferentially followed by micronization.

Alternatively, according to another embodiment, the active substance is micronized and then mixed with the excipients in the form of powder, and the mixture thus obtained is granulated, by wet or dry granulation.

According to another embodiment, the powder used according to the invention may be prepared by spray-drying. The raw materials are solubilized in a solvent and then the resulting solution or suspension is spray-dried. The grain thus obtained may be used directly, or after micronization, for preparing the pharmaceutical or nutraceutical composition administered according to the invention.

The active substance on its own or the final mixture of ingredients may be micronized.

The invention will be more clearly understood using the non-limiting examples described below.

### EXAMPLE 1

### Powders To Be Used According To The Invention

Four powders each having the following weight composition are prepared:

| Composition | Quantity in % |
|---|---|
| Phloroglucinol | 10 |
| Sorbitol | 89 |
| Propylene glycol | 1 |
| Testosterone | 10 |
| Sorbitol | 88 |
| Cremophor RH40 | 2 |
| Dihydrotestosterone | 5 |
| Xylitol | 90 |
| Glycerol | 3 |
| Tween 80 | 2 |
| Molsidomine | 10 |
| Xylitol | 83 |
| Propylene glycol | 5 |
| Montanox 80 | 2 |

The various components are mixed in a mixer/granulator such as ZANCHETTA ROTOLAB mixer/granulator/drier under vacuum, or equivalent, until the mixture is homogenized. A wetting solution or suspension is then incorporated with stirring in order to obtain a wet granule.

This granule is then dried under suitable conditions in order to evaporate the granulation solvent. This granule is then dried and calibrated, and then micronized using an airjet micronization machine of the ALPINE or JETMIL type (or equivalent).

1. Method for administering a pharmaceutical or nutraceutical composition to a subject, said method comprising contacting said pharmaceutical or nutraceutical composition with a mucosal surface, said pharmaceutical or nutraceutical composition containing a powder comprising at least one active substance, at least one surfactant, at least one wetting agent and at least one diluent, whereby rapid and immediate release of the active substance is obtained.

2. Method according to claim 1, wherein at least the active substance is in micronized form.

3. Method according to claim 1, wherein the powder is in micronized form.

4. Method according to claim 1, wherein the active substance is selected from the group consisting of oestradiol and derivatives thereof, norethisterone acetate, progesterone, testosterone, dihydrotestosterone, trinitrine, fentanyl, nitroglycerine, nicotine (nicotine S(−)), scopolamine, clonidine, isosorbide dinitrate, laevonorgestrel in combination with ethinyl oestradiol or with oestradiol, androstanolone, alclometasone dipropionate, acetazolamide, acyclovir, adapalene, alclomethasone dipropionate, amcinonide, ameline, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromicin, flumetasone pivalate, fluocinolon acetonide, fluocinodine, fluocortolone, fluocortolone hexanoate, fluocortolone pivalate, hydrocortisone, hydrocortison acetate, ibacitabine, ibuprofen, imiquimod, ketoconazole, ketoprofen, lidocaine, metronidazole, miconazole nitrate, minoxidil, nifluminic acid, penciclovir, benzoyl peroxide, piroxam, iodinated povidone, promestriene, pyrazinobutazone, roxithromycin, sulphacetamide, triamcinolone, tazarotene, tretinoin and isotretinoin, triclocarban, vidarabine monophosphate, P-3-adrenergic agonist, growth hormone, oxybutinin, buprenorphine, pergolide, oestradiol+nestorone, nestorone, 7α-methyl-19-nortesterone, mecamylamine (nicotine antagonist)+nicotine, salbutamol, selegiline, buspirone, ketotifen, lidocaine, testosterone+oestradiol, ketorolac, eptazocine, insulin, a-interferon, prostaglandins, 17-p-oestradiol+norethindrone acetate, 5-aminolevulinic acid, the benzodiazepine alprozolam, diclofenac, fenoprofen, flubiprofen, ketoprofen, methyl phenidate, miconazole, piroxicam, bruprenorphine, dexmedetomidine, prazosin (α-adrenergic antagonist), gestodene+ethinyl oestradiol, alprostadil, tulobuterol (β-adrenergic agonist), ethinyl oestradiol+norelgestromin, physostigmine, lidocaine, medindolol (α-adrenergic agonist), rotigotine (dopamine D2 antagonist), ethinyl oestradiol+norethindrone acetate, thiatolserine, phlorglucinol, molsidomine, esomeprazole, melagatran (in the case of thrombosis), rosuvastatin, ezetimide, pitavastatin (hyperlipidaemia), mitiglinide (type II diabetes), cilomilast, viozan (asthma), aripipazole (psychiatry), omapatrilat (hypertensive), orzel (cancerology), caspofongin acetate, voriconazole (infections), new COX inhibitors such as etoricoxib (inflammation), valdecoxib (arthritis) and parecoxib, substance P antagonist (depression), darifenacin (urology), eletriptan (migraine), alosetron, tegaserod, capravirine (HIV) and combinations thereof.

5. Method according to claim 1, wherein the active substance is selected from the group consisting of vitamins, mineral salts and brewer's yeast.

6. Method according to claim 1, wherein the surfactant is selected from the group consisting of non-ionic surfactants, such as polyoxyethylene sorbitan (fatty acid ester), polyoxyethylene alkyl ether, the polyoxyethylene derived from castor oil, and mixtures thereof.

7. Method according to claim 1, wherein the wetting agent is selected from the group consisting of polyols such as sorbitol, glycerol or polyethylene glycol, and mixtures thereof.

8. Method according to claim 1, wherein the diluent is selected from the group consisting of sodium, calcium carbonate or bicarbonate, sucrose, mannitol, xylitol, sorbitol, lactose, microcrystalline cellulose or cellulose powder, starch and derivatives thereof, dibasic calcium phosphate, tribasic calcium phosphate, calcium sulphate, dextrates, dextrins, dextrose excipients, fructose, kaolin, lactitol and mixtures thereof.

9. Method according to claim 1, wherein the powder comprises a binding agent selected from the group consisting of acacia, alginic acid, sodium carboxymethylcellulose, microcrystalline cellulose, dextrins, ethylcellulose, gelatin, glucose, guar gum, hydroxypropylmethylcellulose, methylcellulose, polyethylene oxide, povidone, pregelatinized starch, and mixtures thereof.

10. Method according to claim 1, wherein the powder comprises a penetration enhancer selected from the group consisting of aliphatic fatty acid esters such as isopropyl myristate; fatty acids such as oleic acid; alcohols or polyols, such as ethanol, propylene glycol or polyethylene glycol; the components of essential oils and terpene derivatives (such as eugenol, geraniol, nerol, eucalyptol or menthol); surfactants; moisturizers such as glycerol or urea; keratolytic agents, such as alpha-hydroxy acids, 23-lauryl ether, aprotinin, azone, benzalkonium chloride, cetylpyridinium chloride, cetyltrimethylammonium bromide, cyclodextrins, dextran sulphate, lauric acid, lysophosphatidylcholine, menthol, methoxysalicylate, methyl oleate, oleic acid, phosphatidylcholine, polyoxyethylene, polysorbate 80, sodium EDTA, sodium glycocholate, sodium glycodeoxycholate, sodium lauryl sulphate, sodium salicylate, sodium taurocholate, sodium taurodeoxycholate, sulphoxides, alkyl glycosides, and mixtures thereof.

11. Method according to claim 1, wherein the powder has a particle size of between 0.01 μm and 1000 μm.

12. Method according to claim 11, wherein the powder has a particle size between 0.1 μm and 100 μm.

13. Method according to claim 12, wherein the powder has a particle size between 1 μm and 50 μm.

14. Method according to claim 1, wherein the pharmaceutical or nutraceutical composition is applied on the buccal mucous membrane, the nasal mucous membrane or the vaginal mucous membrane.

15. Method according to claim 14, wherein the composition is applied to the buccal mucous membrane sublingually.

16. Method according to claim 1, wherein the composition is in a sprayable form.

17. Method according to claim 1, wherein the composition is contained in a sachet.
